(19) United States
(12) Patent Application Publication
(10) Pub. No.: US 2005/0008622 A1
(43) Pub. Date: Jan. 13, 2005

(54) Clinical application of dendritic cell-based cancer immunotherapy

(76) Inventors: Chi-Dug Kang, Busan (KR); Min-Ho Jeong, Busan (KR)

Correspondence Address: BIRCH STEWART KOLASCH & BIRCH, PO BOX 747, FALLS CHURCH, VA, 22040-0747, US

(21) Appl. No.: 10/495,877

(22) PCT Filed: Nov. 26, 2003
(86) PCT No.: PCT/KR03/02572

(30) Foreign Application Priority Data
Nov. 28, 2002 (KR) 10-2002-0074834

(51) Int. Cl. A61K 48/00

(52) U.S. Cl. 424/93.21

(57) ABSTRACT

The present invention relates to a clinical application of a dendritic cell-based cancer immunotherapy proposed to cure cancers using a dendritic cell, and in particular to a cancer immunity curing method (1) for injecting a dendritic cell into a tumor scanned by a radioactive ray, and a composition (2) which is used as a cancer immunotherapy in such a manner that a dendritic cell that a tumor antigen is loaded by a method of an electroporation, cell fusion or pulsing, is injected into a cancer patient.

### TECHNICAL FIELD

The present invention relates to a cancer immunotherapy, and in particular to a cancer immunotherapy using a dendritic cell that is known as a new type dendritic cell-based cancer immunotherapy implemented based on a method in which a dendritic cell is inoculated into tumor after radioactive rays are scanned in accordance with a known dendritic cell-based cancer immunotherapy using a total cell lysates as a source of antigen, and a method in which an antigen is loaded into a dendritic cell based on an electroporation method.

### BACKGROUND ART

Recently, it is difficult to overcome the basic problems in death due to cancer for the reasons that there are a side effect in a conventional cancer therapy, and a tolerance due to a genetic instability. In the case of recrudescence of cancer, there is not known any therapy.

Therefore, an anti-tumor vaccine development is needed, which is implemented based on an immunity checking system. So far, there are known a combined peptide vaccine, gene vaccine, thermal impact protein vaccine, etc. Among the above methods, it is known that the dendritic cell method is most effective. The dendritic cells are the strongest and important antigen cell in a live body, so that the dendritic cells are well adapted to the anti-tumor vaccine and almost diseases related to the immune reaction.

However, the immunotherapy using the dendritic cell is not standardized. Some research centers are trying to maximize the effects and to implement an easier adjustment. It is well known that the immune system is capable of recognizing tumors and attacking the same. Both humoral immunity and cell-mediated immunity are related to the dissolution of all tumors, but in almost case the regression of the tumors through a particular recognition of the tumor antigen is related to a cell-mediated immunity. In particular, the operations that CD8+ cytotoxic T lymphocyte (CTL) recognizes a tumor cell and kills the same are proved through various models. (1. Boon, T., J. C. Cerottini, B. Van den Eynde, P. van der Bruggen, and A. Van Pel. 1994. Tumor antigens recognized by T lymphocytes. Annu Rev Immunol 12:337. 2. Celluzzi, C. M., J. I. Mayordomo, W. J. Storkus, M. T. Lotze, and L. D. Falo, Jr. 1996. Peptide-pulsed dendritic cells induce antigen-specific CTL-mediated protective tumor immunity. J Exp Med 183, 1:283. 3. Feltkamp, M. C., H. L. Smits, M. P. Vierboom, R. P. Minnaar, B. M. de Jongh, J. W. Drijfhout, J. ter Schegget, C. J. Melief, and V. M Kast 1993.

Vaccination with cytotoxic T lymphocyte epitope-containing peptide protect against a tumor induced by human papillomavirus type 16-transformed cells. Eur J Immunol 23, 9:2242.3). The dendritic cell is a unique antigen-presenting cell that may induce an immunity reaction and is widely used for curing cancer and an antigen-particular immunotherapy of infection diseases. (Banchereau, J., F. Briere, C. Caux, J. Davoust, S. Lebecque, Y. J. Liu, B. Pulendran, and K. Palucka. 2000. Immunobiology of dendritic cells. Annu Rev Immunol 18:767.) The ability that the dendritic cell is initialized so that the T cell recognizes and kills the tumor cells based on the tumor-particular method is reported in various animal models. In addition, the immunization induced based on the dendritic cell may induce an immunity memory for thereby preventing the occurrence of tumors.

The dendritic cells occupies only about 0.3% in the entire circulation leucocyte and is formed of a heterogeneous group having a certain expression type different from macrophage. The dendritic cell is a strong antigen-presenting cell that is largely different from the B cell having a relatively weak antigen-representing ability or macrophage cell. The dendritic cell is induced from a monocytel pulsed by IL-4 and GM-CSF or a monocytel precursor (CD34+ cell) and is used for vaccine protocol. The immaturity dendritic cell extracts apoptotic or necrotic, bacteria and a certain antigen or an antigen epitope from a water soluble protein. The antigens induced into the dendritic cells through various procedures of apoptotic body capture, macropinocytosis, and mannose receptor or endocytosis, etc. through CD32 or CD64 are known to form a complex with a main tissue complex body through a series procedure.

The captured antigen is carried with a MHC class II section and is treated and is proposed as a peptide-MHC complex body for thereby inducing a CD4+ T cell reaction. Recently, it was proved that the dendritic cell has an ability for representing through the MHC I molecular by the proteasome and TAP dependence with respect to the antigen induced from the outside. Therefore, the dendritic cells are judged to pulse the T cell and B cell through a special treatment procedure with respect to an external antigen. The dendritic cell is widely used for the cancer immunity therapy based on the above-described characteristics.

The antigen that is most generally used during the dendritic cell cancer immunotherapy is epitope peptide. However, it is needed to check whether the above antigen is presented in a carcinoma of a patient. The above antigen may be used for only a patient who has a particular HLA type that may form a complex body. In addition, there is not much peptide in the US and Europe. In Korea, it is known that there is not provided the tumor antigen confirmed. Furthermore, the antigens of digestive cancer and respiratory organ those are the targets of the researches of the present inversion are not introduced yet. Searching the tumor antigen needs long time. In Korea, it is impossible to use the peptide antigen for a dendritic cell cancer immunotherapy.

In order to avoid the above problems, there are several trials for using the unidentified TAA (total cell lysates, apoptotic/necrotic cells, total mRNA, tumor cells fused with DC, etc.) as an antigen. However, in this case, there are problems for loading the unidentified TAA on the dendritic cell. The total cell lysates is known to generally pulse CD4+ helper T cell. There is inconvenience for directly separating mRNA from a tumor tissue or cell. The apoptotic cell should be disadvantageously cultivated together with the dendritic cell after a cancer cell is separated and cultivated, and an apoptosis is formed. The confusion of the cancer cell and dendritic cell needs a cancer cell separation cultivation from the tumor tissue. In this research, the above disadvantages are researched in cooperation with the ministry of health and welfare and the Korean science foundation. As a result of the researches, a new method of dendritic cell cancer immunotherapy such as a method for injecting a dendritic cell into tumor to which radioactive rays are scanned and a method for loading an antigen into a dendritic cell based on electroporation is developed.

According to a result of the survey on the 2000 year cancer registration business reported by the ministry of health and welfare and the national cancer center, among the total cancer numbers, the stomach cancer and cervical cancer that are the developing country type cancers are gradually decreased, and the large intestine cancer, breast cancer and lung cancer that are the advanced country type cancers are gradually increased. The total cancer occurrence numbers registered in 2000 is 83,846 showing an increase by 1.9% compared to 1999. Among the cancers, the number of the digestive organ cancer is top. Namely, in the case of male, the stomach cancer, liver cancer and large intestine cancer are 24.5%, 16.3% and 10.2%, respectively, that are the half of the total cancers, and in the case of female, the stomach cancer and large intestine cancer are 15.8% and 10.5%. The lung cancer in the respiratory cancer is significantly increased, and ranks the second place (11.5%) next to the stomach cancer among the total cancers. In the cancer death ratio, the lung cancer is the top.

Even though a cancer earlier detection based on a development of diagnosis technology, an increasing cancer prevention trend and operation, a radioactive curing and anticancer chemical therapy are developed, the cancer occurrence rate and the death ratio due to cancer are not decreased, so that it is urgently needed to induce an improvement in a conventional cancer therapy or a new therapy technique. In the case of a surgical operation or radioactive ray therapy that are a classical type cancer therapy method, a remote micro transit affection may remain. There may be a side effect due to a hematosis function limit may occur due to a destroy of marrow. In the case that a tolerance with respect to a carcinostatis substance of the tumor cell is generated, a desired curing effect is not obtained. Therefore, a method for minimizing the therapy resistance and side effect and preventing a minimal residual lesion is capable of significantly decreasing the death rate due to cancer by preventing a relapse of cancer. In this point, the immunotherapy, namely the dendritic cell-based cancer immunotherapy is known to provide a possibility as the most effective anti-turomr vaccine.

It is intended to introduce the dendritic cell-based cancer immunotherapy based on the present research and to cure patients based on a new method developed by the present researchers.

### DISCLOSURE OF INVENTION

Accordingly it is an object of the present to use a dendritic cell as a cancer immunotherapy agent and to develop a clinical application using the same for curing patient.

Namely, in the present invention, there are developed a method for injecting a dendritic cell into a tumor to which radioactive rays are scanned, and a method for loading an antigen into a dendritic cell based on an electroporation for thereby implementing a new type of dendritic cell-based cancer immunotherapy. In the former method, it is possible to accurately inject a dendritic cell into a tissue of tumor having apoptosis due to radioactive rays, and it is not needed to separate tumor tissues. In the latter method, since it is possible to directly inject a protein antigen into a cytoplasm, so that a CD8+ cytotoxic T cell directly related to kill cancer cells can be more stimulated. In the present invention, it is an object of the present invention to introduce the principle of a conventional dendritic cell-based cancer immunotherapy implemented using a total cell lysates as an antigen source for thereby implementing a clinical application using the same.

### BRIEF DESCRIPTION Of DRAWINGS

The present invention will become better understood with reference to the accompanying drawings which are given only by way of illustration and thus are not limitative of the present invention, wherein;

FIG. 1 is a view illustrating a therapy effect. As shown therein, no therapy effect was observed in the case that only radioactive rays were scanned on a tumor of a right side femoral region when a tumor therapy effect was observed in a group IR in which radioactive rays were scanned to a tumor, and a group (IR+DCs) in which a homogeneous dendritic cell was inoculated after radioactive rays were scanned to a tumor. It was possible to observe a significant therapy effect in the case that a dentritic cell was inoculated into a left side back after radioactive rays were scanned to a tumor of a right side femoral region. The IR group is a group in which only a radioactive ray scanning is performed, and IR+DCs is a group in which a radioactive ray is scanned, and a dendritic cell is induced to the opposite side. The IR group does not have a significant effect. The tumor of the right femoral region is significantly decreased after IR+DCs. Induction of the therapeutic anti-tumor immunity against distance tumor by intratumorial injection of dendritic cells into the irradiated tumor. Photographs (B) were taken after last immunization

In FIG. 2, a solid cancer was provides by its concentration, and the size of growing solid cancer was compared (blue color). In a test group, a solid cancer was formed and was treated by radioactive rays. An immaturity dendritic cell was provided. The size of the tumor of which a growth was inhibited was measured (red color). As a result, a desired effect was obtained. All samples were cured after 6 weeks except for one sample in the test group to which the maximum concentration tumor cell was provided, so that solid cancer was not observed. In the case of the remaining one sample, the average size of 250 mm2 was significantly decreased to the size of 50 mm2.

In FIG. 3, the test was conducted by dividing the test group (IR+DCs) into a tumor (Tm(−)) without tumor, a tumor comparison group (Tm(+)), a dendritic cell treatment group (DCs), a radioactive ray scanned group (IR), and a dendritic cell treatment group after scanning radioactive rays. The most effective cell toxic was shown in the IR+DCs group, so that it was known that a cancer immunotherapy was high. 5:1, 10:1 and 20:1 represent ratios of the Splenocytes:Tumor cells. Enhancement of anti-tumor immunity by intralesional injection of dendritic cells (DC) into irradiated (IR) tumor

### BEST MODE FOR CARRYING OUT THE INVENTION

As a result of the experiments performed by the present researchers, in the cell toxic experiment using a dendritic cell, a maturity dendritic cell stimulates immunity in such a manner that an immaturity dendritic cell is accurately inoculated into a tumor to which radioactive rays are scanned. As a result of the performance experiment with respect to the tumor cells in the immunity by the immunity stimulation, the cell toxic is significantly increased. In addition, in the case that the maturity dendritic cell in which a tumor antigen is loaded externally, it is possible to observe a significant increase of interlukin-2. In addition, the above effect is obtained as a result of the experiment performed with respect to the solid tumor.

### EXAMPLES

The actual experiments according to the present invention will be described in detail.

In the following experiments, the experiment was conducte dusing a dendritic cell as a cancer immunotherpy. As a result of the experiment, the anti-tumor effects and interlukin-2 with respect to the solid cancer are increased.

### Example 1

The MCA102 fibrosarcoma cell was inoculated under the skin of the right femoral region of a syngeneic C57BL/6 mouse (6-8 weeks, female), and the tumor was grown to a diameter of 8 mm. The femoral region having the tumor is placed in the radioactive ray scanning region of the Linac radioactive ray scanning apparatus ((Semens mevatron 67), and then the 15Gy radioactive rays were scanned into the homogeneous dendritic cells in the tumor. The above processes were performed three times per one time per week. After one week, the splenocyte was separated from the mouse of a group a comparison group without tumor, a group with a tumor, a group that the homogenous dendritic cell was injected into the tumor to which the radioactive rays were not scanned, a group that the radioactive rays were scanned to the tumor, and a group that the radioactive rays were scanned, and the homogeneous dendritic cells were scanned, and then the cytotoxicity was measured with respect to the tumor cells. A certain amount of the tumor cells was processed at the magnitude of 5, 10 and 20 times with respect to the splenocytes, and the relative cell toxic was observed.

As a result, in the group having only the tumor, the cytotoxicity was not shown like the comparison group without the tumor. A small amount of the cytotoxicity was shown in the group that the homogenous dendritic cell was injected into the tumor to which the radioactive rays were not scanned, and the group that the radioactive rays were scanned to the tumor. However, in the group that the radioactive rays were scanned and the homogeneous dendritic cells were scanned, the cytotoxicity was significant differently from the other groups. Therefore, it was known that it is possible to significantly increase the anti0tumor immunity by inoculating the dendritic cells into the tumor to which the radioactive rays were scanned (refer to FIG. 3).

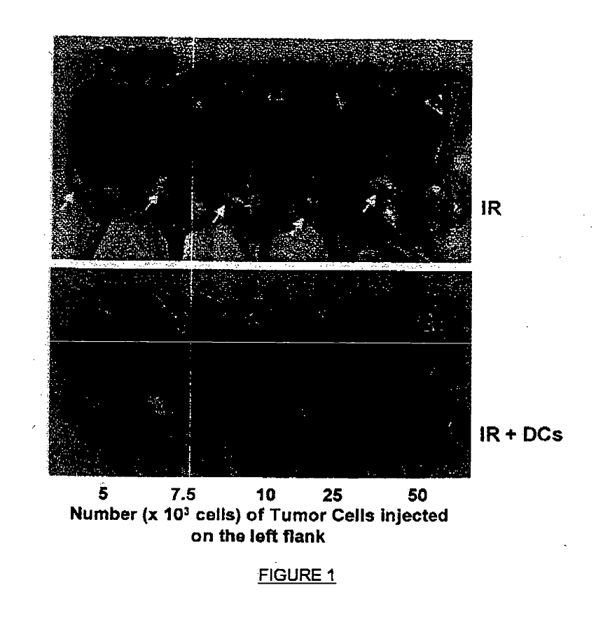

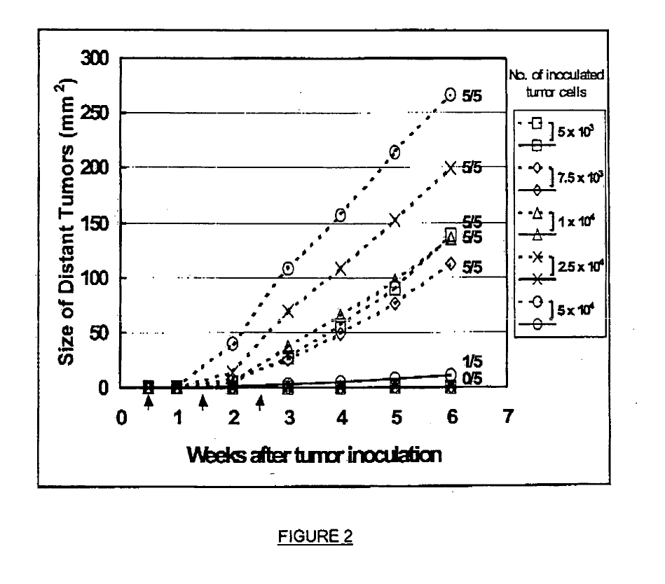

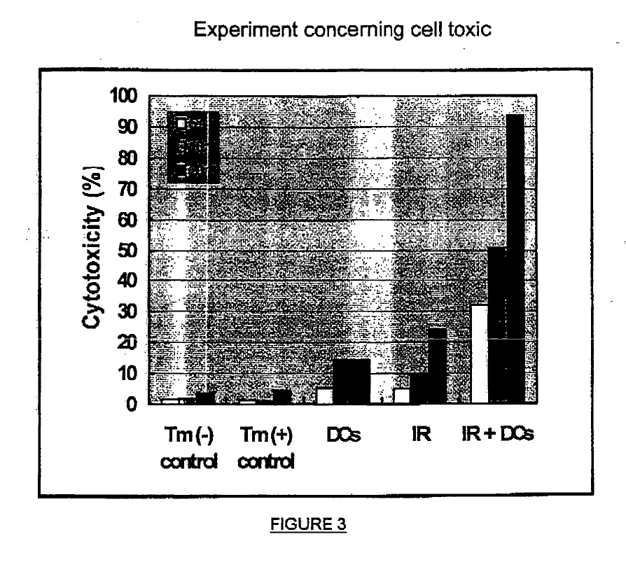

In addition, the tumor therapy effect was checked in the opposite sides in the group that the radioactive rays were scanned to the tumor, and the group that the radioactive rays were scanned, and the homogeneous dendritic cells were inoculated. In the case that be radioactive rays were scanned into the tumor of the femoral region of the right side, the therapy effects were not observed. In the case that the radioactive rays were scanned to the tumor of the right side femoral region, and then the dendritic cells were inoculated, a significant therapy effect was visually observed (refer to FIG. 1). The solid cancers were processed by the concentration, and the sizes of the solid cancers were compared (blue color). In the experiment group, the solid cancers were formed and scanned by the radioactive rays, and then the immaturity dendritic cells were inoculated, and the sizes of the tumors of which the growth was inhibited were compared (red color). As a result, the effects were significant. After six weeks, all mice were cured except for one mouse to which the tumor cells were inoculated in maximum, and the solid cancer was not found. In the case of one mouse still having the tumor, the size of the average 250 mm² were decreased to below 50 mm² (refer to FIG. 2).

### Example 2

There are prepared the group E in which OVA (4 mg/ml) was electroporated, and the OVA was washed and cultivated for 16 hour, the group P in which the OVA (4 mg/ml) was pulsed for 16 hours, and the group E+P that the OVA was electroporated and was pulsed for 16 hours. The IL-2 was measured in the RF33.70 (MHC class I) or D0.11.10 (MHC class II) cells using the dendritic cells of the above three groups. As a result of the measurement, the group E effectively provided the OVA based on the MHC class I. The group E+P provided the OVA to the MHC class I more that the group E (FIG. 1A). As expected, the group P provided more OVA based on the MHC class II compared to the group E, and the group E+P provided more OVA based on the MHC class II compared to the group P (FIG. 1B). As a result, it was possible to more effectively provide the antigen based on the MHC class I by electroporating the antigen to the dendritic cells. It was possible to provide more antigen based on the MHC class II. Therefore, it was expected that the electroporation was capable of effectively increasing the umminity.

[image]

[image]

### Example 3

In a result of the cytotoxicity of the splenocyte with respect to the EG7 cell (EL4 cells transfected with an OVA cDNA) when the OVA was pulsed to the homogeneous dendritic cells based on the electroporation, if the electroporation more effectively presented the OVA based on the MHC class I, when the C57BL/6 mouse was decreased to the OVA-electroporated, it was checked whether a specific immunity was generated compared to when it is decreased based on the OVA-pulsed DC. There were prepare the group E+P that the OVA was electroporated, and the OVA was washed and cultivated for 16 hours, the group that the OVA was pulsed for 16 hours, and the group E+P that the OVA was electroporated, and the the OVA was pulsed for 16 hours. The dendritic cell (1×10⁶ cells) was inoculated under the skin of the right waist of the C57BL/6 mouse totally three times for one time per week. After one week, the splenocyte was separated, and the cell toxic was observed with respect to the EG7 cell (EL4 cells transfected with an OVA cDNA). As a result, the group E had the stronger cell toxic compared to the group P. The cell toxic of the group E+P was stronger than the group E (FIG. 2A). When the above experiment was performed with respect to the EL4 cell, the cell toxic was not observed in all groups (FIG. 2B). As a result, the cell toxic with respect to the EG7 was unique with respect to the OVA. It was known that the electroporaton increased the antigen presentation based on the MHC class I for thereby effectively increasing the immunity.

[image]

The mouse was processed in the above manner. After one week, the EG7 and EL4 cell were inoculated into the left back, and then the tumor occurrence inhibition effect was checked. As a result, it was possible to obtain a certain tumor inhibition effect similar in both the groups E and P. In the group E+P, the tumor occurrence inhibition effects were very high (FIG. 3). In addition, the above experiment was performed with respect to the EL4 cell, the tumor occurrence inhibition effects were not observed in all groups.

[image]

### INDUSTRIAL APPLICABILITY

As described above, in the present invention the cancer immunotherapy using the dendritic cells is effective for curing cancers. The present invention may be well adapted to all kinds of solid cancer therapy using the dendritic cell-based cancer immunotherapy newly developed in the present invention (method for loading the antigen in to the dendritic cell based on electroporation). In the present invention, it is possible to accurately induce an immaturity dendritic cell into an intra-lesion in the tumor to which radioactive rays are scanned with respect to the patients who do not have cancer tissue.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A product containing a dendritic cell and a dendritic cell-based cancer immunotherapy agent containing the same product that are characterized in that a cancer tissue killing agent is loaded into an immaturity dendritic cell with respect to a cancer patient with a cancer tissue for thereby curing cancers.

2. A product prepared for the purpose for curing cancers and a dendritic cell-based cancer immunotherpy agent containing the product that are characterized in that an immaturity dendritic cell is condensed, and a radioactive ray is scanned with respect to a cancer patient who does not have a cancer tissue, and the condensed dendritic cell is inoculated into an intra-lesion formed of apoptosis.

3. A product fabricated for a combined use of dendritic cells loaded with a cancer cell and a dendritic cell-based cancer immunotherapy agent containing the above product that are characterized in that a dendritic cell loaded with a cancer cell is combined by a method of a dendritic cell loaded with a cancer cell and an electroporation method based on a pulse method.

4. A product of a dendritic cell prepared for loading a cancer cell and a dendritic cell-based cancer immunotherapy agent containing the above product that are implemented by performing a pulse operation with respect to a dendritic cell loaded with a cancer cell by a method of electroporation.
